(19) United States
(12) Patent Application Publication
(10) Pub. No.: US 2023/0241353 A1
(43) Pub. Date: Aug. 3, 2023

(54) Skin Nicking Device for Catheter Placement System

(71) Applicant: Bard Access Systems, Inc., (US)

(72) Inventors: Glade H. Howell, Draper, UT (US); Jason R. Stats, Layton, UT (US)

(73) Assignee: Bard Access Systems, Inc., Salt Lake City, UT (US)

(21) Appl. No.: 18/105,743

(22) Filed: Feb. 3, 2023

Related U.S. Application Data
(60) Provisional application No. 63/306,445, filed on Feb. 3, 2022.

(51) Int. Cl. A61M 25/06

(52) U.S. Cl. CPC A61M 25/0612; A61M 25/0606; A61M 25/09

(57) ABSTRACT

A catheter placement system including a catheter having a catheter tube distally coupled to a catheter hub having one or more extension legs proximally coupled to the catheter hub. The catheter tube defines one or more lumens where each of the one or more lumens is in fluid communication with one of the extension legs. The catheter placement system further includes a skin nicking device configured to transition between a sheathed configuration and a deployed configuration. The skin nicking device includes a needle defining a needle lumen, having a needle wall including one or more slots in fluid communication with an outside surface of the needle, and one or more blades configured to be aligned with the one or more slots, the one or more blades coupled to a distal end of one or more connecting members within the needle lumen.

### PRIORITY

This application claims the benefit of priority to U.S. Provisional Application No. 63/306,445, filed Feb. 3, 2022, which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma or increased risk of infection. There is a relatively high potential for touch contamination due to the number of medical devices that need to be interchanged during the Seldinger technique. As such, advanced catheter placement systems have been developed to reduce the number of steps and medical devices involved in placing a catheter, such as a CVC, into a patient.

Some of these advanced catheter placement systems include accessing a vasculature with a needle and stabilizing the access site with a guidewire. Once the guidewire is placed, a scalpel may be used to cut or nick the skin and fascia at the insertion site to ease the insertion of the catheter. If the skin nick is not created properly, a skin bridge may form, impeding insertion of the catheter through the skin into the vessel. A skin nicking device may be used to create a repeatable depth of cut, reducing the likelihood of leaving skin bridges around the insertion site. Disclosed herein are advanced catheter placement systems and associated methods for nicking the skin at the insertion site to eliminate skin bridges impeding the insertion of the catheter into the vasculature.

### SUMMARY

Disclosed herein is a catheter placement device that includes a skin nicking device configured to enlarge an insertion site opening. The skin nicking device, according to some embodiments, includes (i) a needle defining a needle lumen and a needle wall, where the needle includes slot extending through the needle wall; (ii) a blade disposed within the needle lumen in alignment with the slot; and (iii) a connecting member disposed within the needle lumen, where the connecting member is operatively coupled with the blade to facilitate transitioning the blade between a sheathed configuration, wherein the blade is entirely disposed inward of an outside surface of the needle, and a deployed configuration, wherein the blade protrudes radially outward through the slot beyond the outside surface of the needle.

In some embodiments, the blade defines a sharp edge disposed opposite a dull edge, and the blade is oriented within the needle lumen such that the sharp edge is directed radially inward in the sheathed configuration. In some embodiments, the blade is oriented such that the sharp edge is directed distally in the deployed configuration.

In some embodiments, the blade rotates between the sheathed configuration and the deployed configuration, and in some embodiments, the connecting member is slidably disposed within the needle lumen. In some embodiments, the connecting member is coupled with the blade such that longitudinal displacement of the connecting member causes the blade to rotate.

In some embodiments, the connecting member is longitudinally positionable within the needle lumen between a first position and a second position, such that (i) displacement of the connecting member away from the first position toward the second position transitions the blade away from sheathed configuration toward the deployed configuration, and (ii) displacement of the connecting member away from the second position toward the first position transitions the blade away from deployed configuration toward the sheathed configuration. In some embodiments, the first position is proximal the second position.

In some embodiments, the blade is a first blade and the skin nicking device further includes a second blade, the slot is a first slot and the needle further includes a second slot, and the connecting member is a first connecting member and the skin nicking device further includes a second connecting member coupled with the second blade to facilitate transitioning the second blade between (i) the sheathed configuration, wherein the second blade is entirely disposed inward of an outside surface of the needle, and (ii) the deployed configuration, wherein the second blade protrudes radially outward through the second slot beyond the outside surface of the needle. In some embodiments, the second slot is disposed opposite the first slot.

In some embodiments, the first connecting member is configured to rotationally bias the first blade away from the sheathed configuration toward the deployed configuration, and the second connecting member is configured to rotationally bias the second blade away from the sheathed configuration toward the deployed configuration. In some embodiments, the first and second connecting members are attached to the needle wall.

In some embodiments, the needle is configured for insertion within a lumen of a catheter, and the first and second blades are configured to engage a distal end of the catheter when the catheter is distally displaced along the needle such that the first and second blades transition away from the deployed configuration toward the sheathed configuration as the distal end of the catheter travels from a proximal end of the first and second slots toward a distal end of the first and second slots.

Also disclosed herein is a catheter assembly that, according to some embodiments, includes (i) a catheter including a catheter tube proximally coupled to a catheter hub having one or more extension legs proximally coupled therefrom, where the catheter tube defines one or more lumens and where each of the one or more lumens is in fluid communication with one of the extension legs; and (ii) the catheter placement device according to any of the embodiments described above. In some embodiments of the assembly, the needle of the catheter placement device is inserted into one of the one or more lumens of the catheter tube.

Also disclosed herein is a method of placing a catheter within a blood vessel of a patient that, according to some embodiments, includes (i) inserting a needle of a skin nicking device through a skin and into the blood vessel to define an insertion site, where a blade of the skin nicking device is transitionable between a sheathed configuration and a deployed configuration, and where the blade extends radially away from an outside surface of the needle in the deployed configuration; (ii) nicking the skin with the blade to enlarge the insertion site; and (iii) inserting a catheter through the insertion site into the blood vessel.

In some embodiments, the further includes transitioning the blade from the sheathed configuration to the deployed configuration, and in some embodiments of the method, transitioning the blade from the sheathed configuration to the deployed configuration includes rotating the blade.

In some embodiments of the method, the skin nicking device includes a connecting member disposed within a needle lumen of the needle, where the connecting member is operatively coupled the blade, and where the connecting member is configured to transition the blade between the sheathed configuration and the deployed configuration.

In some embodiments of the method, longitudinal displacement of the connecting member causes rotation of the blade.

In some embodiments of the method, in the sheathed configuration an entirety of the blade disposed radially inward of the outside surface of the needle.

In some embodiments of the method, the connecting member is longitudinally positionable within the needle lumen between a first position and a second position, and transitioning the blade from a sheathed configuration to the deployed configuration includes displacing the connecting member from the first position to the second position.

In some embodiments of the method, the blade is rotatably coupled with the needle wall.

In some embodiments, the method further includes transitioning the blade from the deployed configuration to the sheathed configuration.

In some embodiments of the method, (i) the blade is a first blade and the skin nicking device further includes a second blade, (ii) the slot is a first slot and the needle further includes a second slot, and (iii) the connecting member is a first connecting member and the skin nicking device further includes a second connecting member coupled with the second blade to facilitate transitioning the second blade between the sheathed configuration, where the second blade is entirely disposed inward of an outside surface of the needle, and the deployed configuration, where the second blade protrudes radially outward through the second slot beyond the outside surface of the needle.

In some embodiments of the method, the first connecting member is configured to rotationally bias the first blade away from the sheathed configuration toward the deployed configuration, and the second connecting member is configured to rotationally bias the second blade away from the sheathed configuration toward the deployed configuration.

In some embodiments, the method further includes distally advancing the catheter along the needle to engage the first and second blades with a distal end of the catheter, wherein the engagement causes the first and second blades to transition away from the deployed configuration toward the sheathed configuration.

Also disclosed herein is a manufacturing method of a catheter placement device that, according to some embodiments, includes (i) forming a slot extending through a needle wall of a needle, (ii) placing a blade within a needle lumen of the needle adjacent the slot such that a sharp edge of the blade is directed radially inward with respect to the needle, (iii) placing a connecting member within the needle lumen; and (iv) coupling the connecting member with the blade.

In some embodiments of the manufacturing method, coupling the connecting member with the blade includes rotatably coupling the connecting member with the blade. In some embodiments, the manufacturing method further includes rotatably coupling the blade with the needle wall.

In some embodiments of the manufacturing method, coupling the connecting member with the blade includes fixedly attaching the connecting member to the blade. In some embodiments, the manufacturing method further includes fixedly attaching the connecting member to the needle wall.

In some embodiments, the manufacturing method further includes (i) forming a second slot extending through the needle wall, (ii) placing a second blade within the needle lumen adjacent the second slot such that a sharp edge of the second blade is directed radially inward with respect to the needle, (iii) placing a second connecting member within the needle lumen, (iv) fixedly attaching the second connecting member to the second blade, and (v) fixedly attaching the second connecting member to the needle wall.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system in an unfolded configuration, in accordance with embodiments disclosed herein;

FIG. 1B shows a plan view of the catheter placement system of FIG. 1A in a folded configuration ready for use, in accordance with embodiments disclosed herein;

FIG. 1C shows a perspective view of a catheter placement system of FIG. 1A in a folded configuration, in accordance with embodiments disclosed herein;

FIG. 2 shows a side view of a catheter of a catheter placement system of FIG. 1A in an unfolded configuration, in accordance with embodiments disclosed herein;

FIG. 3A shows close up detail of a distal portion of the catheter of FIG. 2, in accordance with embodiments disclosed herein;

FIGS. 3B-3C show cross-section views of the catheter of FIG. 3A, in accordance with embodiments disclosed herein;

FIG. 4 shows a longitudinal cross-section view of a distal portion of the catheter placement system of FIG. 1A, in accordance with embodiments disclosed herein;

FIGS. 5A-5E show various cross-section side views of the catheter placement system of FIG. 1A illustrating an exemplary method of use, in accordance with embodiments disclosed herein;

FIG. 6A illustrates a cross-sectional side view of a first embodiment of a skin nicking device in a deployed configuration, in accordance with some embodiments;

FIG. 6B illustrates a cross-sectional side view of the skin nicking device of FIG. 6A in a sheathed configuration, in accordance with some embodiments;

FIG. 7A illustrates a cross-sectional side view of a second embodiment of a skin nicking device in a deployed configuration, in accordance with some embodiments;

FIG. 7B illustrates a cross-sectional side view of the skin nicking device of FIG. 7A in a partially transitioned configuration, in accordance with some embodiments;

FIG. 7C illustrates a cross-sectional side view of the skin nicking device of FIG. 7A in a sheathed configuration, in accordance with some embodiments;

FIG. 7D illustrates a cross-sectional end view of the skin nicking device of FIG. 7A in the sheathed configuration, in accordance with some embodiments;

FIG. 7E illustrates a cross-sectional view of the skin nicking device of FIG. 7A in the deployed configuration, in accordance with some embodiments;

FIG. 8 illustrates a flow chart of an exemplary method of placing a catheter using a skin nicking device, in accordance with some embodiments; and

FIG. 9 illustrates a flow chart of an exemplary method of manufacturing a skin nicking device, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

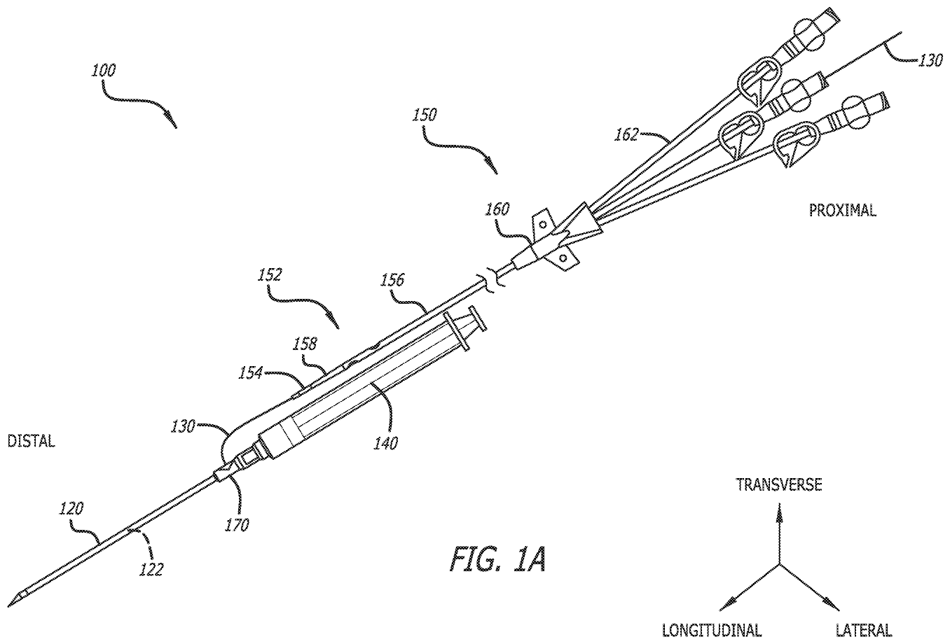

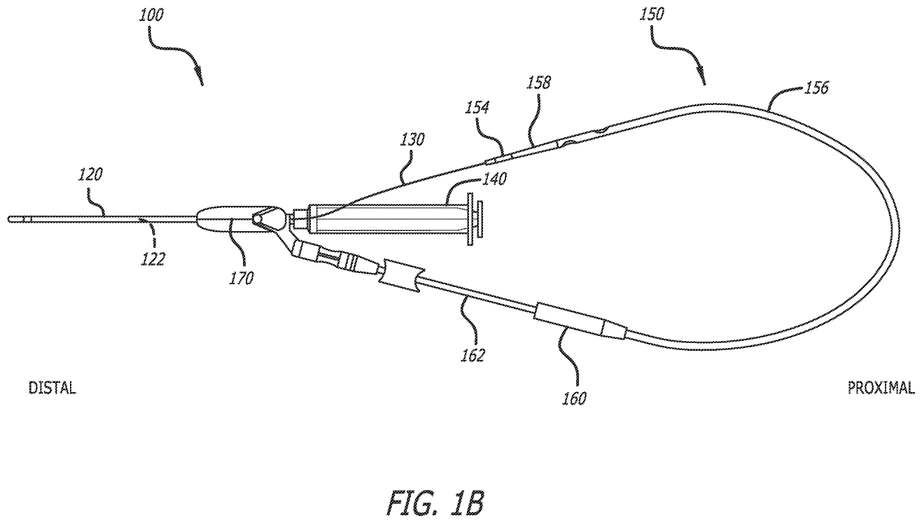

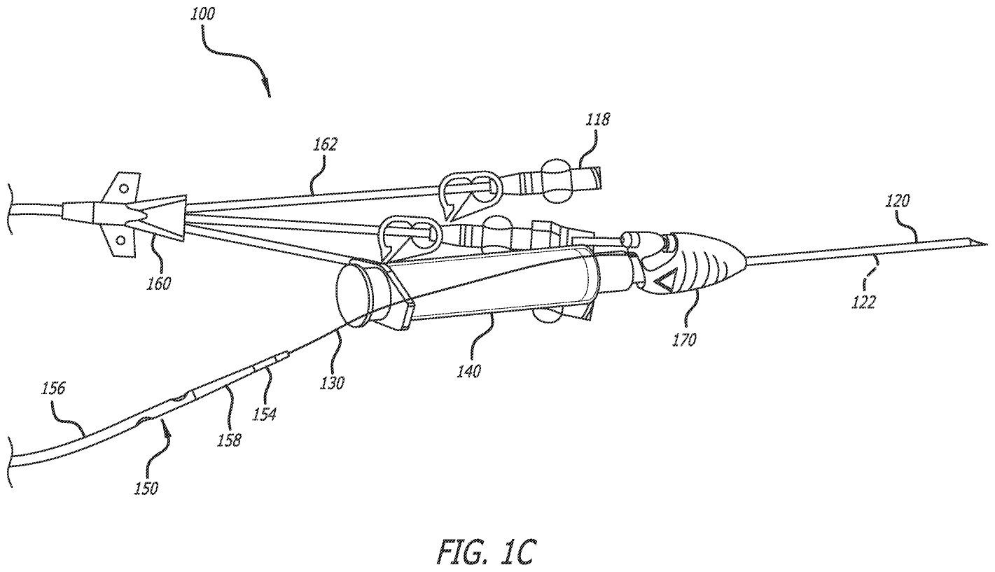

FIGS. 1A-1C show an exemplary advanced catheter placement system (“system”) 100, generally including a needle 120, a guidewire 130, a syringe system 140, a catheter 150, and a needle housing (“housing”) 170. FIG. 1A shows the system 100 in an unfolded configuration for ease of illustration. FIG. 1B shows a plan view of the system 100 in a folded configuration ready for use. FIG. 1C shows a perspective view the system 100 in a folded configuration. In an embodiment the catheter placement system 100 can be a Rapidly Insertable Central Catheter (RICC) placement system 100 configured to place a RICC 150. However, it will be appreciated that other catheter placement systems configured to place other types of catheters are also contemplated. Exemplary catheters 150 can also include peripheral intravenous (PIV) catheters, peripherally inserted central catheter (PICC), central venous catheters (CVC), midline catheters, dialysis catheters, single lumen catheters, multi-lumen catheters, or the like.

In an embodiment, the catheter 150 can generally include a catheter body 152 supported at a proximal end by a catheter hub (“hub”) 160. The hub 160 can include one or more extension legs 162 extending proximally therefrom. Each extension leg of the one or more extension legs 162 can be in fluid communication with a lumen of the catheter body 152. The catheter body 152 can include a first section 154 disposed distally, a second section 156 disposed proximally, and a transition section 158 disposed therebetween. The first section 154 can define a single lumen and have a first outer diameter, the second section 156 can define two or more lumen and can have a second diameter larger than the first diameter. The transition section 158 disposed between the first section 154 and the second section 156 can define a tapered shape extending from the first diameter of the first section to the second diameter of the second section. A guidewire 130 can extend through a lumen of the catheter 150 from a proximal end of an extension leg 162, to a distal tip of the first section 154.

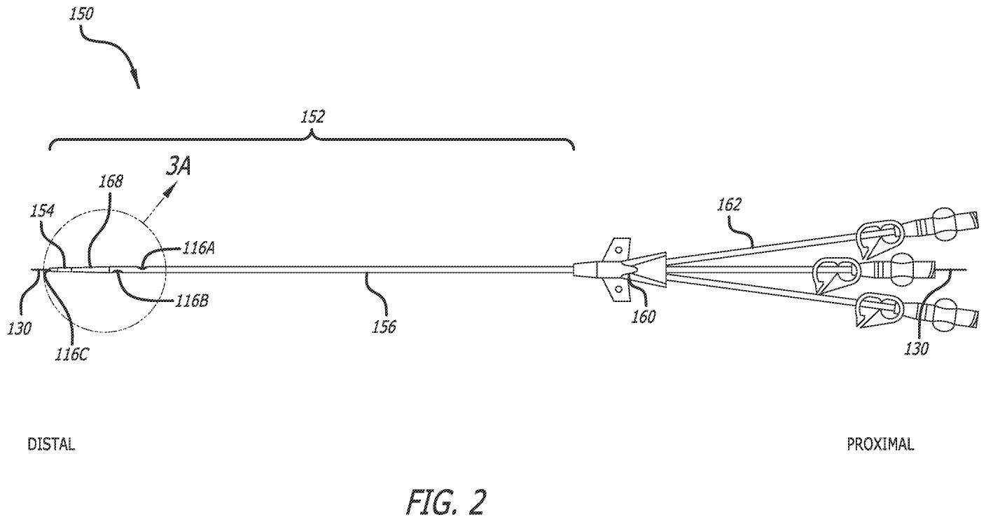

FIG. 2 shows further details of an exemplary catheter 150 of the system 100. As described herein, different sections of the catheter 150 are required to perform different functions and as such are required to display different mechanical properties. For example, the first section 154 and the transition section 158 can provide a more rigid mechanical properties or harder durometer material relative to the second section 156. As such, the first section 154 and transition section 158 can withstand greater axial forces without kinking or collapsing, as theses sections are urged distally, forming and dilating the insertion site. The second section 156 can be formed of a softer durometer, or a more compliant material to facilitate negotiating the second section 156 through tortuous vascular pathways.

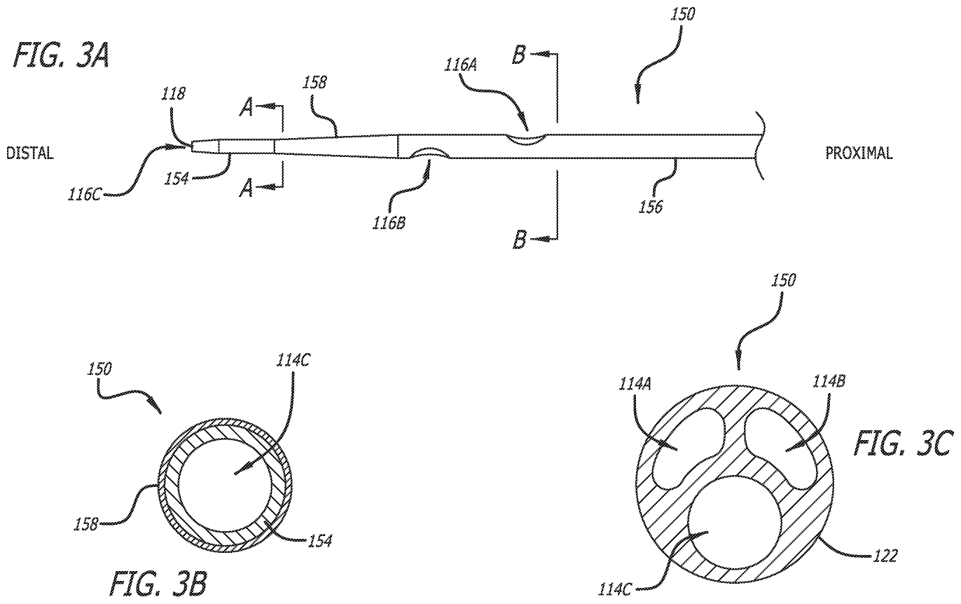

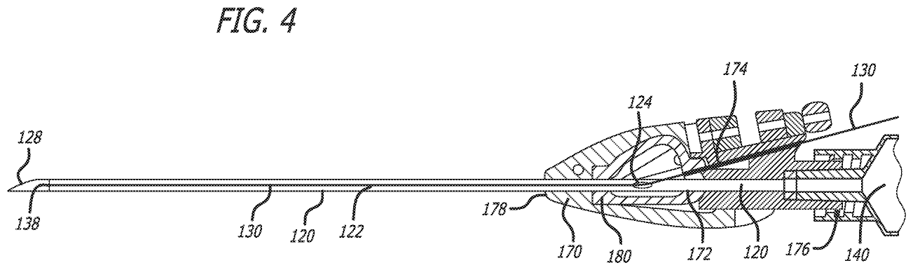

FIGS. 3A-3C show further details of a distal portion of the catheter 150, including the first section 154, the second section 156, and the transition section 158. In an embodiment, the second section 156 can include a proximal lumen 114A terminating at a proximal lumen aperture 116A, and a medial lumen 114B terminating at a medial lumen aperture 116B. Each of the proximal lumen aperture 116A and the medial lumen aperture 116B can extend through a side wall of the second section 156. Each of the proximal lumen aperture 116A and the medial lumen aperture 116B can be disposed proximally of the transition section 158. The proximal lumen aperture 116A can be disposed proximally of the medial lumen aperture 116B.

FIG. 3B shows a cross section view of the catheter body 152 at point “A” of FIG. 3A. As shown, the first section 154 can define a single lumen and a relatively smaller outer diameter. In an embodiment, a proximal portion of the first section 154 can be received within a distal portion of the transition section 158. A distal lumen 114C of the catheter 150 can extend to a distal tip 118 of the catheter 150 and can communicate with a distal lumen aperture 116C. FIG. 3C shows a cross section view of the second section 156 at point “B” of FIG. 3A, showing the proximal lumen 114A, medial lumen 114B and distal lumen 114C.

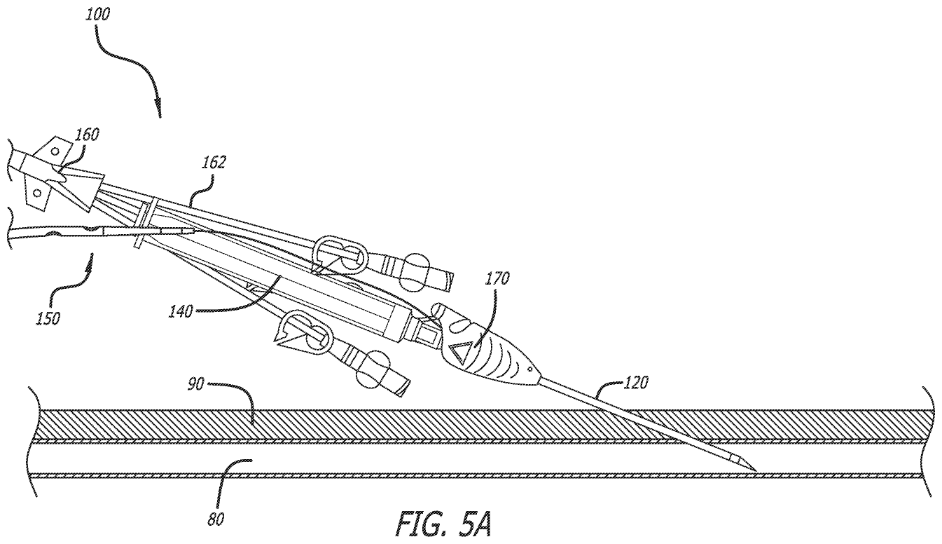

FIG. 4 shows a longitudinal cross-section view of a distal portion of a catheter placement system 100 including the needle 120, guidewire 130, a distal portion of the syringe system 140, and needle housing (“housing”) 170 including a needle splitter system 180, as described in more detail herein. In an embodiment, a proximal end of the needle 120 can be supported by a needle hub which can be coupled to, and supported by, a distal end of the syringe system 140. The syringe system 140 can be in fluid communication with needle lumen 122. The syringe system 140 can be configured to form a vacuum therein and draw a fluid flow proximally through the needle lumen 122. In an embodiment, the needle 120 can include a guidewire aperture 124 disposed in a wall of the needle 120 and communicating with a needle lumen 122. A distal portion of the guidewire 130 can extend through the guidewire aperture 124 and into the needle lumen 122. In an embodiment, a distal tip 138 of the guidewire 130 can be disposed proximate a distal tip 128 of the needle 120. As such, once the needle 120 accesses the vasculature, the distal tip 138 of the guidewire 130 can be positioned within the vasculature, expediting the placement of the catheter 150.

In an embodiment, the catheter placement system 100 can include a housing 170. The housing 170 can include a housing lumen 172 extending between a proximal end 176 and a distal end 178 of the housing 170. The housing 170 can further include a guidewire lumen 174 communicating with the housing lumen 172 and extending at an angle therefrom. A portion of the needle 120 can slidably engage the housing lumen 172. Further, the proximal end 176 of the housing can releasably engage one or both of a needle hub and a distal portion of the syringe system 140. When the housing 170 is engaged with the syringe system 140 the guidewire aperture 124 of the needle 120 can align with the guidewire lumen 174 of the housing 170. As such, the guidewire 130 can extend through the guidewire lumen 174 of the housing 170, through the guidewire aperture 124 of the needle 120 and into the needle lumen 122.

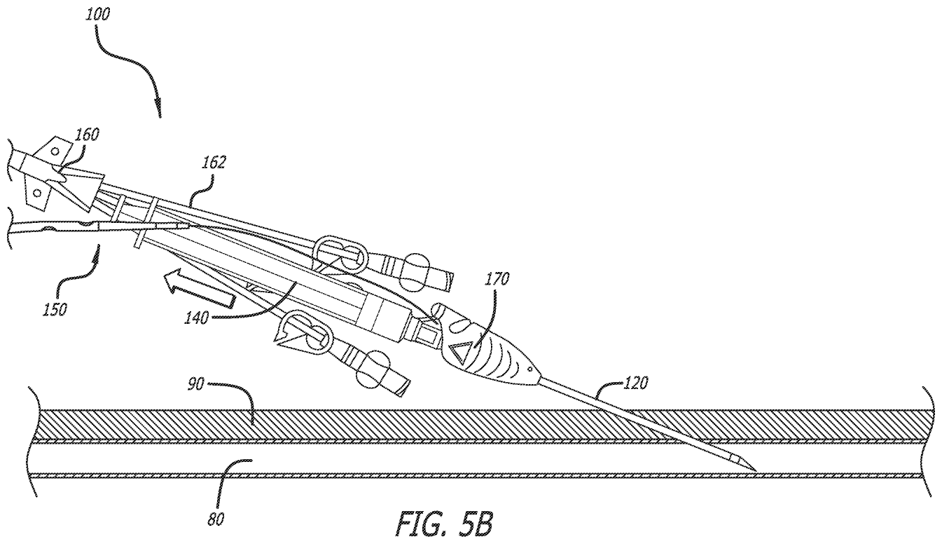

FIGS. 5A-5E show an exemplary method of placing a catheter 150 using the catheter placement system 100. As shown in FIG. 5A, the needle 120 can penetrate surface tissues 90 of the patient and access a vasculature 80, forming an insertion site. As shown in FIG. 5B, a syringe system 140, or similar device can form a vacuum and draw a fluid flow proximally through a needle lumen 122. A user can observe a color or pulsatile flow and confirm correct vascular access. Where incorrect vascular access is confirmed, the needle 120 can be withdrawn and the insertion site can be closed. As shown in FIG. 5C, once correct vascular access has been confirmed, the guidewire 130 can then be advanced through the needle lumen 122 and into the vasculature 80 to maintain patency of the insertion site.

As shown in FIG. 5D, the needle 120 and syringe system 140 assembly can be withdrawn proximally to disengage the needle 120 from the guidewire 130 while leaving a distal portion of the guidewire 130 in place within the vasculature 80. As described in more detail herein, the housing 170 can include a splitter system 180 configured to split the needle 120 longitudinally, as the needle 120 is withdrawn proximally. A portion of the guidewire 130 can pass between the two halves of the needle 120 to allow the needle 120 to disengage the guidewire 130.

As shown in FIG. 5E, with the needle 120 and syringe system 140 assembly disengaged from the guidewire 130, the catheter 150 can then be advanced over the guidewire 130 and into the vasculature. The first section 154 of the catheter 150, having only a single lumen and defining a relatively smaller outer diameter, can enter the vasculature 80 over the guidewire 130, anchoring the insertion site. The transition section 158 can then be urged distally, dilating the insertion site to allow the relatively larger diameter second section 156, defining two or more lumen, to enter the vasculature 80. Once the catheter 150 has been placed, the guidewire 130 can be withdrawn proximally.

Further details and embodiments of such catheter placement systems 100 can be found, for example, in U.S. Pat. No. 10,376,675, US 2019/0255294, US 2021/0069471, US 2021/0085927, US 2021/0113809, US 2021/0113810, US 2021/0121661, US 2021/0121667, US 2021/0228843, US 2021/0322729, US 2021/0330941, US 2021/0330942, US 2021/0361915, US 2021/0402153, US 2021/0402149, US 2022/0001138, U.S. patent application Ser. No. 17/390,682 filed Jul. 30, 2021, and U.S. Provisional Patent Application No. 63/229,862 filed Aug. 5, 2021, each of which is incorporated by reference in its entirety into this application.

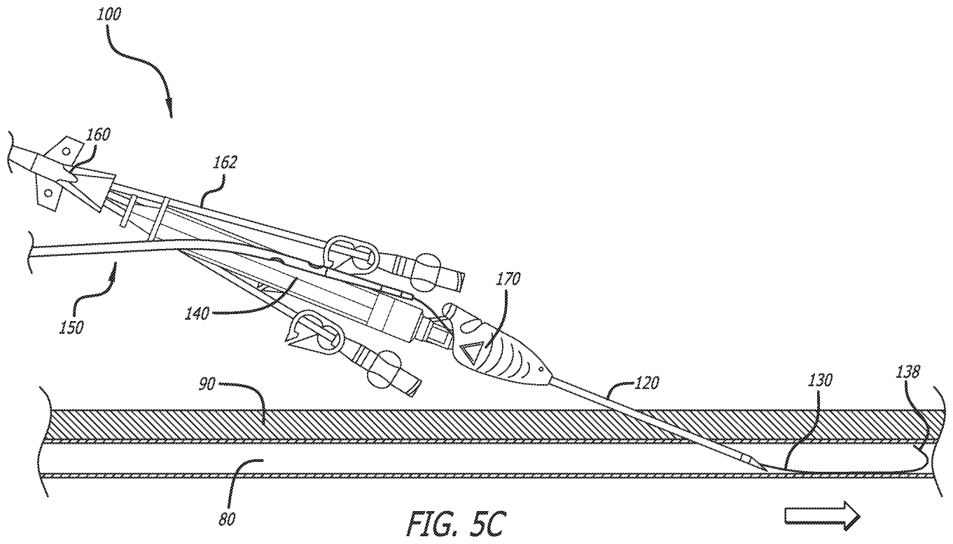

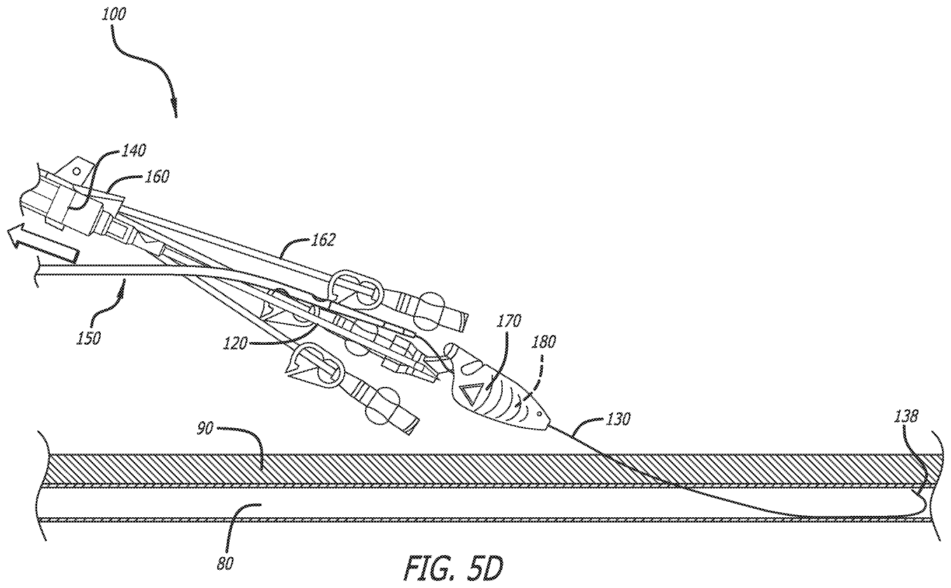

FIGS. 6A and 6B illustrate a cross-sectional side views of a skin nicking device 210 in deployed configuration and a sheathed configuration, respectively, in accordance with some embodiments. As illustrated in FIG. 6A, skin nicking device 210 may include the needle 220 having a slot 223 extending through a needle wall 221 between a needle lumen 222 and an outside surface 225 of the needle 220.

The skin nicking device 210 includes a blade 214 coupled to a connecting member 212 at a distal end thereof, where connecting member 212 is disposed within the needle lumen 222. The connecting member 212 is generally configured to extend an entire length of the needle 220. The connecting member 212 is generally configured to apply a pushing force and/or a pulling to the blade 214 in response manipulation of a proximal portion of the connecting member 212. In some embodiments, the connecting member 212 may be rigid, such as a stylet, for example. In other embodiments, the connecting member 212 flexible in bending, such as a guidewire, for example. In some embodiments, the connecting member 212 may include one or more rigid portions and one or more flexible portions.

The blade 214 is rotatably coupled to the needle 220 at a pivot point 215. The connecting member 212 is coupled with the blade 214 at an attachment point 213, where attachment point 213 is radially offset from the pivot point 215 so that longitudinal displacement of the connecting member 212 causes the blade 214 to rotate about the pivot point 215. As such, the connecting member 212 is configured to transition (i.e., rotate) the blade 214 between the deployed configuration as shown in FIG. 6A and the sheathed configuration as shown in FIG. 6B. In the illustrated embodiment, distal displacement of the connecting member 212 causes the blade 214 to transition away from the sheathed configuration toward the deployed configuration. Conversely, proximal displacement of the connecting member 212 causes the blade 214 to transition away from the deployed configuration toward the sheathed configuration. In other embodiments, skin nicking device 210 may be configured such that distal displacement of the connecting member 212 causes the blade 214 to transition toward the sheathed configuration and proximal displacement of the connecting member 212 causes the blade 214 to transition toward the deployed configuration. FIG. 6A shows the connecting member 212 in a second position consistent the blade 214 disposed in the deployed configuration. Similarly, FIG. 6B shows the connecting member 212 in a first position consistent the blade 214 disposed in the sheathed configuration. In the illustrated embodiment, the second position is distal the first position. In other embodiments, the second position may be proximal the first position.

The blade 214 includes a sharp edge 216 and a dull edge 218. In some embodiments, as illustrated in FIG. 6A, in the deployed configuration, the blade 214 may extend radially away from the outside surface 225 such that the sharp edge 216 faces distally, i.e., toward a distal end 229 of the needle 220, and the dull edge 218 faces proximally, i.e., toward a proximal end 227 of the needle 220. In some embodiments, the blade 214 may protrude perpendicularly away from the outside surface 225. In the deployed configuration, the blade 214 extends through the slot 223. In some embodiments, in the deployed configuration, the dull edge 218 may contact the needle wall 221 (e.g., at a proximal end of the slot 223) to provide a stabilizing force (e.g., prevent further rotation) for the blade 214 as the needle 220 is inserted into the vasculature 80. In the deployed configuration, the sharp edge 216 of the blade 214 may contact a portion of skin or surface tissues 90 to cut through the skin (e.g., nick the skin) as the needle 220 is inserted into the vasculature 80, thereby enlarging the insertion site to accommodate a catheter.

In some embodiments, when the blade 214 is in the sheathed configuration, the entirety of the blade 214 may be disposed within the needle lumen 222. In some embodiments, in the sheathed configuration as illustrated in FIG. 6B, the dull edge 218 of each of the blade 214 may extend longitudinally along the slot 223 within the needle lumen 222. In some embodiments, the connecting member 212 may be manually displaced by the user to transition the blade 214 between the sheathed configuration and the deployed configuration. In some embodiments, the connecting member 212 may be manually actuated through a lever, a spring, or the like (not shown). In an embodiment, the blade 214 may be configured to only transition from the sheathed configuration to the deployed configuration and not vice versa. In some embodiments, the blade 214 may be rotatable biased about the pivot point 215 toward the deployed configuration. For example, the blade 214 may be coupled with the needle wall 221 via a torsional spring (not shown), where the torsional spring applies a biasing torque to the blade 214. In other embodiments, the blade 214 may rotatably biased about the pivot point 215 toward the sheathed configuration.

In some embodiment, the skin nicking device 210 may be provided with the introducer catheter 150, including coupled with the introducer catheter 150 to define a catheter assembly. In some embodiments, the catheter assembly may include the needle 220 disposed within a lumen of the introducer catheter 150.

In some embodiments, the may be provided in the sheathed configuration. As such, in use, the skin nicking device 210 may be transitioned from the sheathed configuration to the deployed configuration.

According to another embodiment, the blade 214 may be rotatably coupled to only the connecting member 212 such that the blade 214 may be longitudinally displaced within the needle lumen 222 together with the connecting member 212. In such an embodiment, the blade 214 may be rotatably biased with respect to the connecting member 212 toward the deployed configuration. In use, the blade 214 may be displaced between a proximal position proximal the slot 223 and a distal position adjacent the slot 223. In the proximal position, the blade 214 is contained within the lumen 222 so as to define the sheathed configuration. As the blade 214 is distally displaced toward the distal position, the biasing force may rotate the blade 214 so that the blade 214 enters and passes through the slot 223. When the blade 214 is disposed in the distal position, the biasing force may transition/rotate the blade 214 fully to the deployed configuration. In some embodiments, the blade 214 and/or the needle 220 may be configured to constrain the blade 214 in alignment with the slot 223. In an embodiment, the needle wall 221 of the opposing side from the slot 223 may include an indent/channel configured to receive therein a portion of the blade 214. The indent may be configured to constrain the blade 214 in alignment with the slot 223.

In some embodiments, the skin nicking device 210 may include a blade barrier 250 protruding inward from the needle wall 221 into the needle lumen 222. In some embodiments, the blade barrier 250 may be located distal the slot 223. In some embodiments, the blade barrier 250 may be located on the needle wall 221 on the same side as the slot 223 or may be located on the needle wall 221 opposing the slot 223. The blade barrier 250 may be configured to limit distal displacement of the blade 214 through the needle lumen 222.

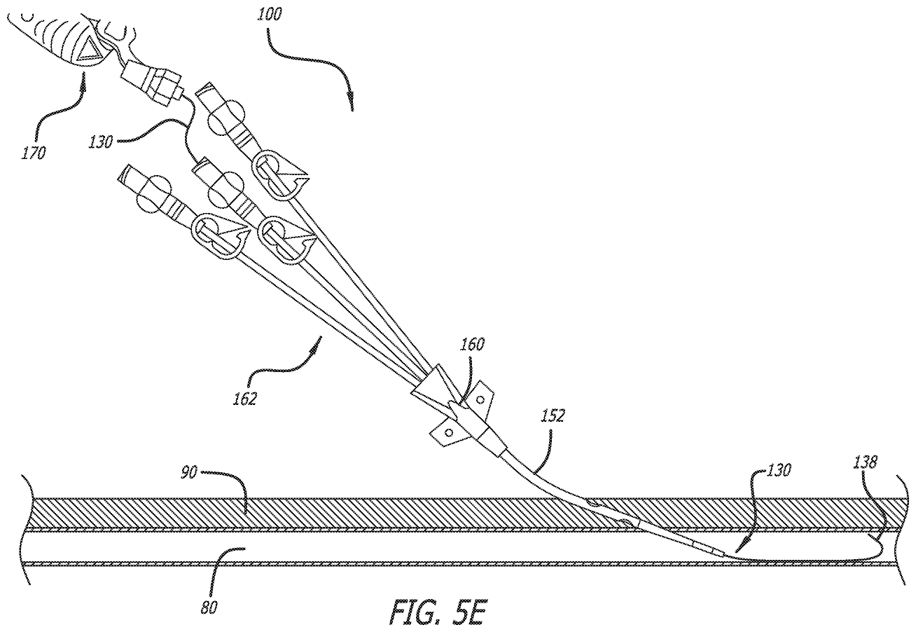

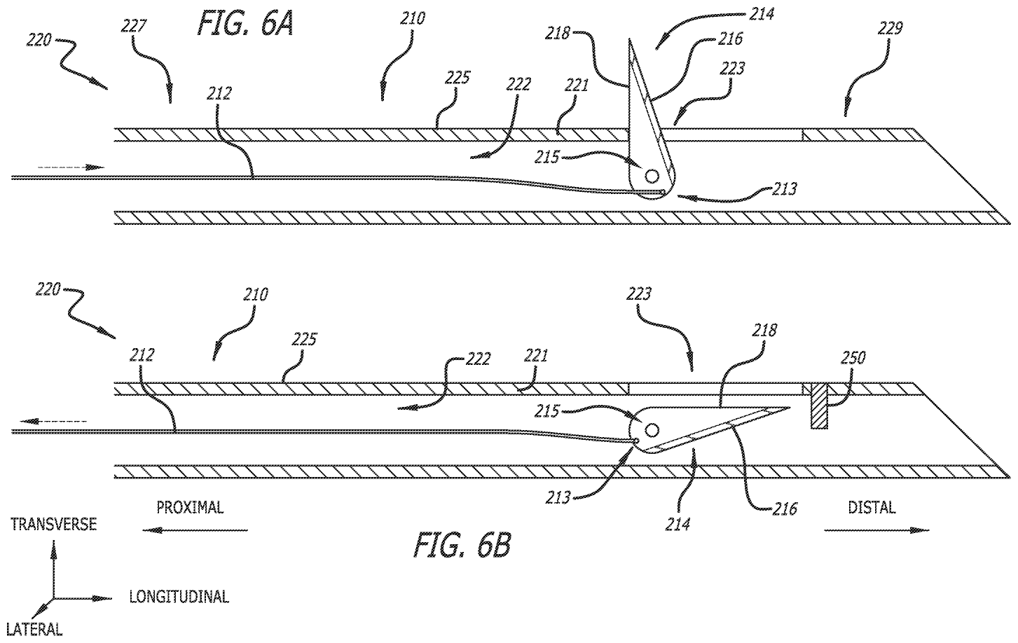

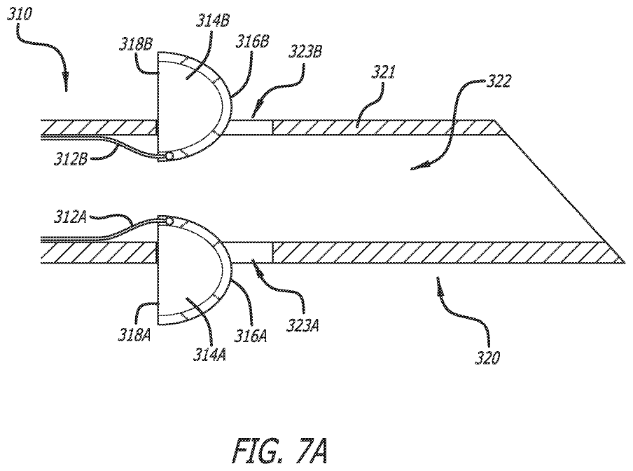

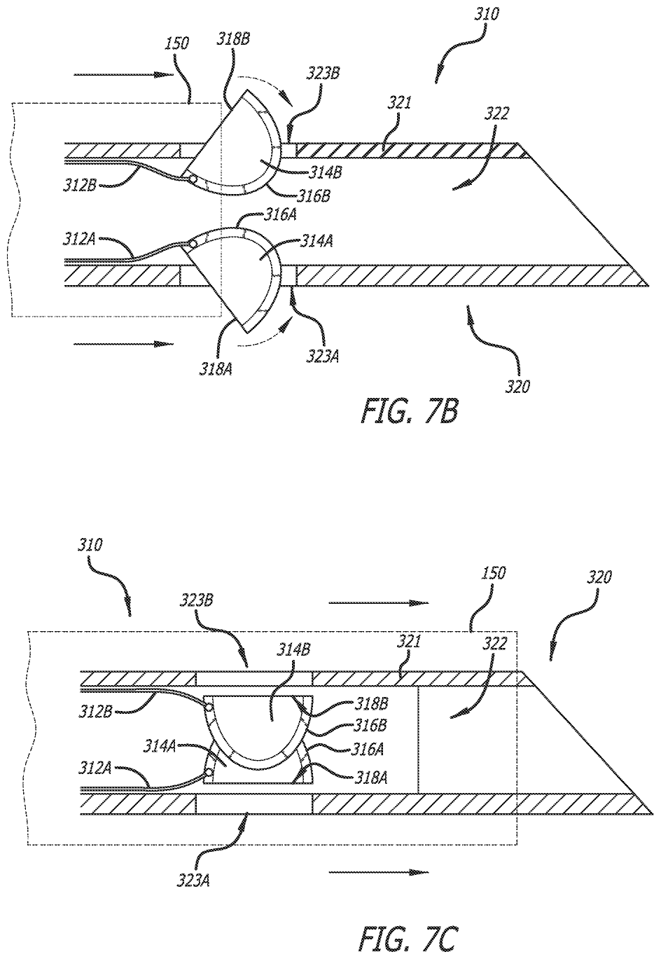

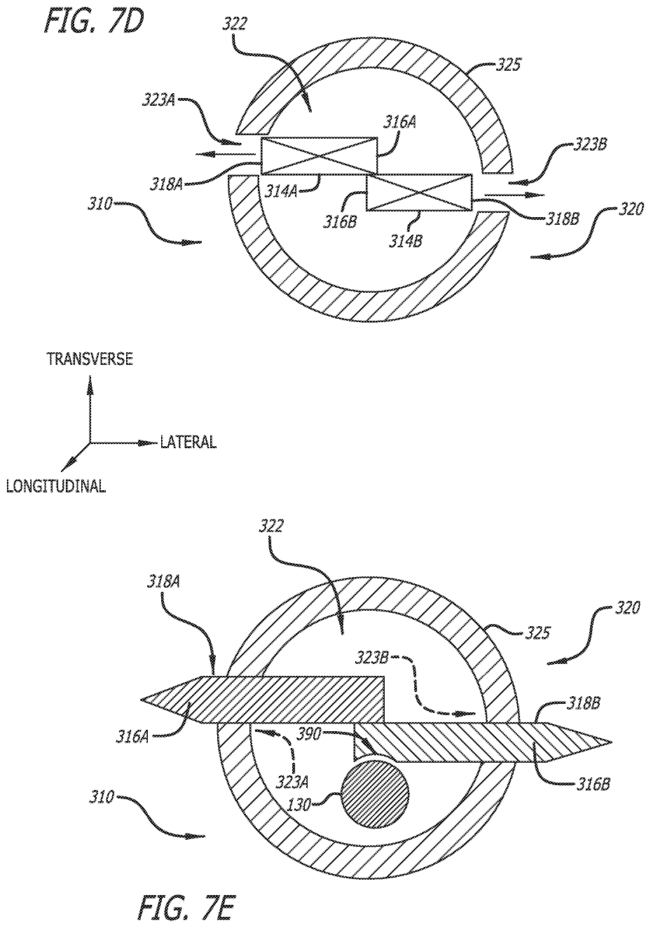

FIGS. 7A-7E illustrate another embodiment of a skin nicking device 310 that can, in certain respects, resemble components of the skin nicking device 210 described in connection with FIGS. 6A-6B. It will be appreciated that all the illustrated embodiments may have analogous features. Accordingly, like features are designated with like reference numerals, with the leading digits incremented to “3.” For instance, the needle is designated as “220” in FIGS. 6A-6B, and an analogous needle is designated as “320” in FIGS. 7A-7E. Relevant disclosure set forth above regarding similarly identified features thus may not be repeated hereafter. Moreover, specific features of the skin nicking device 210 and related components shown in FIGS. 6A-6B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the skin nicking device 310 of FIGS. 7A-7E. Any suitable combination of the features, and variations of the same, described with respect to the skin nicking device 210 and components illustrated in FIGS. 6A-6B can be employed with the skin nicking device 310 and components of FIGS. 7A-7E, and vice versa.

FIG. 7A illustrates a cross-sectional view of the skin nicking device 310 in the deployed configuration. In the illustrated embodiment, the skin nicking device 310 includes two blades 314A/314B extending radially/laterally away from the needle 320 in opposite directions. In other embodiments, the skin nicking device 310 may include only one blade or more than 2 blades. The two blades 314A/314B extend through the needle wall 321 via two corresponding slots 323A/323B, respectively. The two blades 314A/314B are coupled with two corresponding connecting members 312A/312B that extend proximally along the needle lumen 222. The two connecting members 312A/312B are configured to transition the two blades 314A/314B from the sheathed configuration to the deployed configuration. In the illustrated embodiment, the connecting members 312A/312B define a biasing force/torque applied to the blades 314A/314B toward the deployed configuration. In some embodiments, the connecting members 312A/312B (e.g., a proximal end thereof) may be coupled with (e.g., attached to) the needle wall 321. The connecting members 312A/312B may be formed any material suitable for applying the biasing force/torque to the blades 314A/314B, such as stainless steel, or Nitinol, for example.

In the illustrated embodiment, in the deployed configuration, the sharp edges 316A/316B of the blades 314A/314B may be oriented distally with the dull edges 318A/318B of the blades 314A/314B oriented proximally. In some embodiments, with the skin nicking device 310 in the deployed configuration, the skin nicking device 310 may be inserted into the vasculature 80 and may be configured to nick the skin and surface tissues 90 as the skin nicking device 310 is inserted into the vasculature 80, thereby enlarging the insertion site to accommodate a catheter.

FIG. 7B illustrates the skin nicking device 310 in a state of transition between the deployed configuration and the sheathed configuration. As illustrated in FIG. 7B, the introducer catheter 150 may be placed over the needle 320, and the introducer catheter 150 may be displaced distally and/or proximally along the needle 320. The introducer catheter 150 may engage the blades 314A/314B (e.g., the dull edges 318A/318B) to transition the blades 314A/314B away from the deployed configuration toward the sheathed configuration. More specifically, engagement of the introducer catheter 150 with the blades 314A/314B may cause the blades 314A/314B rotate and/or displace inward through the slots 323A/323B and into the needle lumen 222 in opposition to the biasing force of the connecting members 312A/312B. As the blades 314A/314B are rotated inward, the sharp edges 316A/316B may be oriented radially inward so that the dull edges 318A/318B are oriented outward so as to face the slots 323A/323B. The rotation/displacement of the blades 314A/314B inward towards the needle lumen 322 transitions the skin nicking device 310 from the deployed configuration to the sheathed configuration.

In some embodiment, the skin nicking device 310 may be provided with the introducer catheter 150, including coupled with the introducer catheter 150 to define a catheter assembly. In some embodiments, the catheter assembly may include the needle 320 disposed within a lumen of the introducer catheter 150. In some embodiments of the catheter assembly, the introducer catheter 150 may extend over the slots 323A/323B to constrain the blades 314A/314B in the sheathed configuration. As such, in use, the introducer catheter 150 may be displaced proximally along the needle 220 to transition the blades 314A/314B from the sheathed configuration to the deployed configuration.

FIG. 7C illustrates the skin nicking device 310 in the sheathed configuration. As shown, the introducer catheter 150 is distally displaced beyond the slots 323A/323B such that the blades 314A/314B are constrained within the lumen 222 by the introducer catheter 150, thereby defining the sheathed configuration. As shown, the blades 314A/314B are rotated so as to be contained within the needle lumen 322.

FIGS. 7D-7E illustrate a cross-sectional end views of the skin nicking device 310 transitioning between the sheathed configuration (FIG. 7D) and the deployed configuration (FIG. 7E). In some embodiments, the blades 314A/314B may be laterally and/or transversely offset as illustrated in FIG. 7D. In the sheathed configuration, the blades 314A/314B may be rotated and contained within the needle lumen 322. The blades 314A/314B may be rotated so that the dull edges 318A/318B face the slots 323A/323B. In some embodiments, when the blades 314A/314B are laterally offset and/or transversely offset, the sharp edges 316A/316B may be disposed adjacent or in physical contact with each other within the needle lumen 322, as illustrated in FIG. 7D.

In the deployed configuration (FIG. 7E), the blades 314A/314B may rotate outward from the needle lumen 322 through the corresponding slots 323A/323B. When the blades 314A/314B rotate outward, the sharp edges 316A/316B are oriented towards the distal end of the needle 320 so that the sharp edges 316A/316B may nick the skin and surface tissues 90 as the needle 320 is inserted through the skin and into the blood vessel. The dull edges 318A/318B are oriented toward the proximal end of the needle 320. In some embodiments, whether the blades 314A/314B are in the sheathed configuration and/or deployed configuration, the blades 314A/314B may occupy a portion of the cross-sectional area of the needle lumen 322. Although the blades 314A/314B may occupy a portion of the cross-sectional area of the needle lumen 322, the needle lumen 322 may be configured to receive additional devices (e.g., guidewire 130) therein. In some embodiments, the guidewire 130 may be configured to move through the needle lumen 322 alongside the blades 314A/314B. In an embodiment, at least one of the blades 314A/314B may include a channel 390 thereon, where the channel 390 is configured to allow passage of the guidewire 130 through the needle lumen 322. The channel 390 may be oriented (i) parallel to the guidewire 130 when the blades 314A/314B are in the sheathed configuration, or (ii) parallel to the guidewire when the blades 314A/314B are in the deployed configuration as illustrated in FIG. 7E. In some embodiments, the blades 314A/314B and/or the needle 320 may be configured to constrain the blades 314A/314B on a lateral plane defined by the slots 323A/323B.

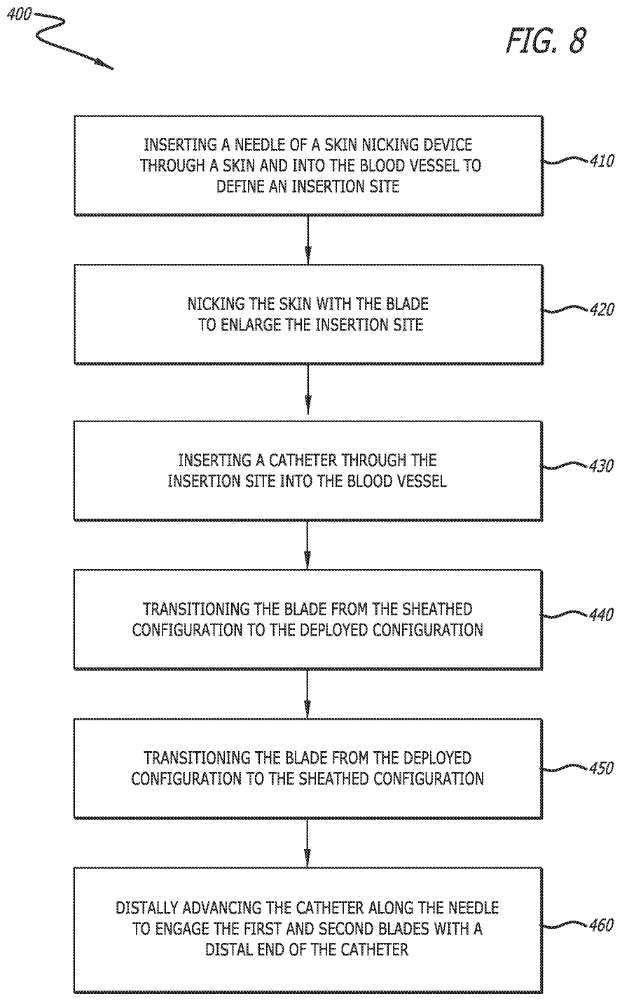

FIG. 8 illustrates a flow chart of an exemplary method 400 of placing a catheter within a blood vessel of a patient that includes all or any subset of the following steps, actions, or processes. The method 400 may include inserting the needle of the skin nicking device through the skin and into the blood vessel to define an insertion site (block 410), where the blade of the skin nicking device is transitionable between a sheathed configuration and a deployed configuration, and where the blade extends radially away from an outside surface of the needle in the deployed configuration. In some embodiments of the method 400, in the sheathed configuration, an entirety of the blade disposed radially inward of the outside surface of the needle.

The method 400 may further include nicking the skin with the blade to enlarge the insertion site (block 420). In some embodiments of the method 400, the skin nicking device includes the connecting member disposed within the needle lumen of the needle, where the connecting member is operatively coupled the blade within the needle lumen such that longitudinal displacement of the connecting member causes rotation of the blade. In some embodiments of the method 400, the blade is rotatably coupled with the needle wall. The method 400 may further include inserting a catheter through the insertion site into the blood vessel (block 430).

The method 400 may further transitioning the blade from the sheathed configuration to the deployed configuration (block 440). In some embodiments of the method 400, transitioning the blade from the sheathed configuration to the deployed configuration includes rotating the blade. In some embodiments of the method 400, the connecting member is longitudinally positionable within the needle lumen between a first position and a second position, and transitioning the blade from a sheathed configuration to the deployed configuration includes displacing the connecting member from the first position to the second position.

In some embodiments, the method 400 may further include transitioning the blade from the deployed configuration to the sheathed configuration (block 450).

In some embodiments of the method 400, the blade is a first blade and the skin nicking device further includes a second blade. Further, the slot is a first slot and the needle further includes a second slot, and further still, the connecting member is a first connecting member and the skin nicking device further includes a second connecting member coupled with the second blade to facilitate transitioning the second blade between (i) the sheathed configuration, where the second blade is entirely disposed inward of an outside surface of the needle, and (ii) the deployed configuration, where the second blade protrudes radially outward through the second slot beyond the outside surface of the needle.

In some embodiments of the method 400, the first connecting member is configured to rotationally bias the first blade away from the sheathed configuration toward the deployed configuration, and the second connecting member is configured to rotationally bias the second blade away from the sheathed configuration toward the deployed configuration.

In some embodiments, the method 400 further includes distally advancing the catheter along the needle to engage the first and second blades with a distal end of the catheter (block 460), wherein the engagement causes the first and second blades to transition away from the deployed configuration toward the sheathed configuration.

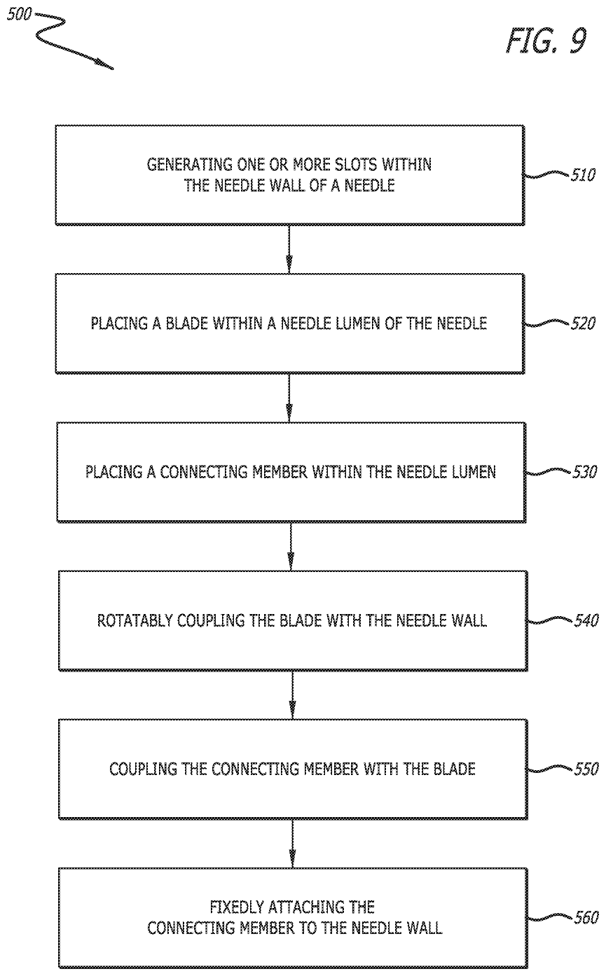

FIG. 9 illustrates a flow chart of an exemplary manufacturing method 500 of a catheter placement device that includes all or any subset of the following steps, actions, or processes. The manufacturing method 500 may include forming a slot extending through the needle wall of the needle (block 510).

The manufacturing method 500 may further include placing the blade within a needle lumen of the needle (block 520), where the blade is disposed adjacent the slot such that the sharp edge of the blade is directed radially inward with respect to the needle.

The manufacturing method 500 may further include placing a connecting member within the needle lumen (block 530). In some embodiments of the method 500, connecting member may extend along proximally along the needle beyond a proximal end of the needle. The manufacturing method 500 may further include rotatably coupling the blade with the needle wall (block 540).

The manufacturing method 500 may further include coupling the connecting member with the blade (block 550). In some embodiments of the manufacturing method 500, coupling the connecting member with the blade includes rotatably coupling the connecting member with the blade. In some embodiments of the manufacturing method 500, coupling the connecting member with the blade includes fixedly attaching the connecting member to the blade. The manufacturing method 500 may further include fixedly attaching the connecting member to the needle wall (block 560).

In some embodiments, the manufacturing method 500 further includes (i) forming a second slot extending through the needle wall, (ii) placing a second blade within the needle lumen adjacent the second slot such that a sharp edge of the second blade is directed radially inward with respect to the needle, (iii) placing a second connecting member within the needle lumen, (iv) fixedly attaching the second connecting member to the second blade, and (v) fixedly attaching the second connecting member to the needle wall.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement device, comprising:
a skin nicking device configured to enlarge an insertion site opening, comprising:
a needle defining a needle lumen and a needle wall, the needle including a slot extending through the needle wall;
a blade disposed within the needle lumen in alignment with the slot; and
a connecting member disposed within the needle lumen, the connecting member operatively coupled with the blade to facilitate transitioning the blade between:
a sheathed configuration, wherein the blade is entirely disposed inward of an outside surface of the needle, and
a deployed configuration, wherein the blade protrudes radially outward through the slot beyond the outside surface of the needle.

2. The device according to claim 1, wherein:
the blade defines a sharp edge disposed opposite a dull edge, and
the blade is oriented within the needle lumen such that the sharp edge is directed radially inward in the sheathed configuration.

3. The device according to claim 1, wherein the blade is oriented such that the sharp edge is directed distally in the deployed configuration.

4. The device according to claim 1, wherein the blade rotates between the sheathed configuration and the deployed configuration.

5. The device according to claim 1, wherein the connecting member is slidably disposed within the needle lumen.

6. The device according to claim 5, wherein the connecting member is coupled with the blade such that longitudinal displacement of the connecting member causes the blade to rotate.

7. The device according to claim 6, wherein:
the connecting member is longitudinally positionable within the needle lumen between a first position and a second position, such that:
displacement of the connecting member away from the first position toward the second position transitions the blade away from sheathed configuration toward the deployed configuration, and
displacement of the connecting member away from the second position toward the first position transitions the blade away from deployed configuration toward the sheathed configuration.

8. The device according to claim 7, wherein the first position is proximal the second position.

9. The device according to claim 1, wherein:
the blade is a first blade and the skin nicking device further includes a second blade,
the slot is a first slot and the needle further includes a second slot, and
the connecting member is a first connecting member and the skin nicking device further includes a second connecting member coupled with the second blade to facilitate transitioning the second blade between:
the sheathed configuration, wherein the second blade is entirely disposed inward of an outside surface of the needle, and
the deployed configuration, wherein the second blade protrudes radially outward through the second slot beyond the outside surface of the needle.

10. The device according to claim 9, wherein the second slot is disposed opposite the first slot.

11. The device according to claim 9, wherein:
the first connecting member is configured to rotationally bias the first blade away from the sheathed configuration toward the deployed configuration, and
the second connecting member is configured to rotationally bias the second blade away from the sheathed configuration toward the deployed configuration.

12. The device according to claim 9, wherein the first and second connecting members are attached to the needle wall.

13. The device according to claim 9, wherein:
the needle is configured for insertion within a lumen of a catheter, and
the first and second blades are configured to engage a distal end of the catheter when the catheter is distally displaced along the needle such that the first and second blades transition away from the deployed configuration toward the sheathed configuration as the distal end of the catheter travels from a proximal end of the first and second slots toward a distal end of the first and second slots.

14. A catheter assembly, comprising:
a catheter including a catheter tube distally coupled to a catheter hub having one or more extension legs proximally coupled thereto, the catheter tube defining one or more lumens, wherein each of the one or more lumens is in fluid communication with one of the extension legs; and
the catheter placement device according to any of the preceding claims, the catheter placement device coupled with the catheter.

15. The assembly according to claim 14, wherein the needle of the catheter placement device is inserted into one of the one or more lumens of the catheter tube.

16-28. (canceled)

29. A manufacturing method of a catheter placement device, comprising:
forming a slot extending through a needle wall of a needle;
placing a blade within a needle lumen of the needle adjacent the slot such that a sharp edge of the blade is directed radially inward with respect to the needle;
placing a connecting member within the needle lumen; and
coupling the connecting member with the blade.

30. The manufacturing method according to claim 29, wherein coupling the connecting member with the blade includes rotatably coupling the connecting member with the blade.

31. The manufacturing method according to claim 29, further comprising rotatably coupling the blade with the needle wall.

32. The manufacturing method according to claim 29, wherein coupling the connecting member with the blade includes fixedly attaching the connecting member to the blade.

33. The manufacturing method according to claim 32, further comprising fixedly attaching the connecting member to the needle wall.

34. The manufacturing method according to claim 32, further comprising:
forming a second slot extending through the needle wall;
placing a second blade within the needle lumen adjacent the second slot such that a sharp edge of the second blade is directed radially inward with respect to the needle;
placing a second connecting member within the needle lumen;
fixedly attaching the second connecting member to the second blade; and
fixedly attaching the second connecting member to the needle wall.
